(19) United States
(12) Patent Application Publication
(10) Pub. No.: US 2020/0075153 A1
(43) Pub. Date: Mar. 5, 2020

(54) GEOFENCED EQUIVALENCE RECOMMENDATIONS FOR MEAL PLAN MENU

(71) Applicant: NUTRISTYLE INC., Meridian, ID (US)

(72) Inventors: Scott Murdoch, Bend, OR (US); Todd Albro, Eagle, ID (US); Caleb Skinner, Beaverton, OR (US); Shannon Madsen, Livermore, CA (US); Lee Brillhart, Seattle, WA (US)

(21) Appl. No.: 16/557,976

(22) Filed: Aug. 30, 2019

Related U.S. Application Data
(60) Provisional application No. 62/725,343, filed on Aug. 31, 2018.

(51) Int. Cl. G16H 20/60; G06F 3/0482; H04W 4/021; G06F 16/909; H04W 4/029; H04W 64/00; G06Q 10/10

(52) U.S. Cl. CPC G16H 20/60; G06F 3/0482; H04W 4/021; G06Q 10/1093; H04W 4/029; H04W 64/003; G06F 16/909

(57) ABSTRACT

A method of generating geofenced equivalence recommendations for a menu generating system involves communicating spatiotemporal location for a user device from a user interface (UI) wizard at a geofenced equivalence recommendations algorithm, configuring the geofenced equivalence recommendations algorithm with user location preferences comprising a food source type parameter, a food source distance parameter, and proximal food preferences received from the UI wizard, identifying a geographic region and a geolocation from the spatiotemporal location through operation of the geofenced equivalence recommendations algorithm, configuring a food component selector with the geographic region, the geolocation, and the user location preferences to identify relevant proximal food databases, and configuring a menu generation algorithm with the geographic region, the geolocation, and the proximal food preferences to generate at least one spatiotemporal location based meal entry in a meal plan menu from the relevant proximal food databases.

### BACKGROUND

Adhering to a diet with caloric and nutrient goals, and that is optimized with respect to other user preferences, including food brands and grocers, is especially challenging when travelling or when in an unfamiliar place. The unfamiliarity of a new location may make it difficult for individuals to find local restaurants or grocery stores that have the foods that will meet their goals, are close enough to the user, and offer local cuisine that fits the user's diet. Therefore, a need exists for an improved way of meeting dietary goals while travelling, or to take account of the fact, for example, that a user may have access to a significantly different set of, for example, food, meal/snacks, brand, grocer options, and restaurant menus consistent with meal plan parameters when at home and when at work.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates a system 100 in accordance with some embodiments.

FIG. 2 illustrates a method 200 in accordance with some embodiments.

FIG. 3 illustrates a system 300 in accordance with some embodiments.

FIG. 4 illustrates a system 400 in accordance with some embodiments.

FIG. 5 illustrates a system 500 in accordance with some embodiments.

FIG. 6 illustrates a system 600 in accordance with some embodiments.

FIG. 7 illustrates a system 700 in accordance with some embodiments.

FIG. 8 illustrates a system 800 in accordance with some embodiments.

FIG. 9 illustrates a system 900 in accordance with some embodiments.

FIG. 10 illustrates a simplified system 1000 in which a server 1004 and a client device 1006 are communicatively coupled via a network 1002 in accordance with some embodiments.

FIG. 11 is an example block diagram of a computing device 1100 that may incorporate embodiments of the present invention in accordance with some embodiments.

### DETAILED DESCRIPTION

“Food” refers to any substance consumed to provide nutritional support for an organism. For example, foods may be an assortment of consumable substances that include meats, grains, dairy products, fruits, mushrooms, vegetables, any plants, animals, insects, microbes, and any isolated or modified component of these. The foods may include condiments such as spices that may be added in combination to the aforementioned foods. Furthermore, foods may include beverages. Individual foods may be combined as components of a meal.

“Meal” refers to a single food component or combination of food components served individually or in combinations as a dish. A meal may include a dish of a variety of food components and spices accompanied by a beverage.

“Nutrient” refers to a substance used by an organism to survive, grow, and reproduce. The requirement for dietary nutrient intake applies to animals, plants, fungi, and protists. Nutrients can be incorporated into cells for metabolic purposes or excreted by cells to create non-cellular structures, such as hair, scales, feathers, or exoskeletons. Some nutrients can be metabolically converted to smaller molecules in the process of releasing energy, such as for carbohydrates, lipids, proteins, and fermentation products (ethanol or vinegar), leading to end-products of water and carbon dioxide. Nutrients include both macronutrients and micronutrients. Macronutrients provide energy and are chemical compounds that humans consume in the largest quantities and provide bulk energy are classified as carbohydrates, proteins, and fats. Water must be also consumed in large quantities. Micronutrients support metabolism and include dietary minerals and vitamins. Dietary minerals are generally trace elements, salts, or ions such as copper and iron. Some of these minerals are essential to human metabolism. Vitamins are organic compounds essential to the body. They usually act as coenzymes or cofactors for various proteins in the body. Nutrients also include bioactive compounds and nutraceuticals, which may be compounds found in foods, are not necessarily synthesized by the body, and are not directly involved in any fundamental functions of the body, yet can alter various metabolic functions within the body to impact health or disease. Some of these nutrients may include lipoic acid, ubiquinones (e.g., CoQ10, carotenoids, phenolic compounds, and the like). Other nutrients impact the functional characteristics of foods, which is defined by how the nutrients impact the consumer. For example, foods of this type include nutrients which impact the glycemic index/load which determines the impact of the food in causing increased blood glucose and/or insulin levels and acid/alkali forming which focuses on the impact on pH levels in the blood and cells, for example.

“Food Distributor” refers to any purveyor (e.g., grocery store, grocery delivery service, etc.) that primarily offers ingredients to a user to utilize as the components of a meal, with the unit size of the ingredient being greater than the quantity required for an individual meal portion. A main difference between a food distributor and a restaurant/food service is in the quantity of the components usually exceeding the quantity required for a single meal.

The phrases “in one embodiment”, “in various embodiments”, “in some embodiments”, and the like are used repeatedly. Such phrases do not necessarily refer to the same embodiment. The terms “comprising”, “having”, and “including” are synonymous, unless the context dictates otherwise.

Reference is now made in detail to the description of the embodiments as illustrated in the drawings. While embodiments are described in connection with the drawings and related descriptions, there is no intent to limit the scope to the embodiments disclosed herein. On the contrary, the intent is to cover all alternatives, modifications and equivalents. In alternate embodiments, additional devices, or combinations of illustrated devices, may be added to or combined, without limiting the scope to the embodiments disclosed herein.

A method of generating geofenced equivalence recommendations for a menu generating system involves communicating the spatiotemporal location, for a user device accessing a user interface (UI) wizard, to a geofenced equivalence recommendations algorithm, configuring the geofenced equivalence recommendations algorithm starting with preferences in the user's profile (including, e.g., food preferences, restrictions, health objectives, budget, preferred brands, grocers or food distributors, and kcal target), and adding user location preferences comprising a food source type parameter, a food source distance parameter, and proximal food preferences received from the UI wizard, identifying a geographic region and a geolocation from the spatiotemporal location through operation of the geofenced equivalence recommendations algorithm, configuring a food component selector with the geographic region, the geolocation, and the user location preferences to identify relevant proximal food databases, and configuring a menu generation algorithm with the geographic region, the geolocation, the proximal food preferences, and user meal preferences to generate at least one spatiotemporal location based meal entry in a meal plan menu from the relevant proximal food databases.

In some configurations, the geofenced equivalence recommendations algorithm may be initially configured with user meal preferences from in the user's profile (including, e.g., food preferences, restrictions, health objectives, budget, preferred brands, grocers or food distributors, and kcal target), as a source for initial configurations.

In some configurations, the user meal preferences comprises food dislikes, food likes, food allergies or restrictions, meal and snack preferences (including preferred recipes), nutrient targets, weight or other personal health objectives, preferred food brands, and grocer or food distributor preferences, kcal target and meal presets. The linked user services may comprise a food tracking service and a grocery list service. In some examples, the user meal preferences comprise a food acquisition option, which may include any of a number of various ways of acquiring food. For example, some food acquisition options include a food delivery service, dining at a restaurant, a grocery store, vending machines, or available inventory in a user's pantry, among others.

In some configurations, a user may provide presets defining which food items to utilize for some food sub categories, identify the food item as a meal component and/or part of a larger food component category. The presets may also be utilized to identify specific food items as well as particular combinations of food items that may be viewed as individual meals by themselves.

In some configurations, a user profile may include user preferences such as food preferences (e.g., likes/dislikes, which may be further broken down into preferred tastes and/or textures, smells, etc.), restrictions (e.g., allergies or disease), health objectives (e.g., lose weight), financial budget, grocer or food distributor (e.g., grocer supplier or direct-to-consumer provider in certain food distribution scenarios), preferred brands or private labels, preferred recipes, and preferred restaurants.

In some configurations, the method of generating geofenced equivalence recommendations for a menu generating system may also include receiving the geolocation of the mobile device through the mobile device's location services at the geofenced equivalence recommendations algorithm, and identifying the geographic region from the geolocation through operation of the geofenced equivalence recommendations algorithm.

Furthermore, in some instances, the method of generating geofenced equivalence recommendations for a menu generating system may receive the spatiotemporal location from a user's calendar service linked with the menu generating system.

In some configurations, the method of generating geofenced equivalence recommendations for a meal plan may utilize a set up assistant (wizard) to configure a geofenced equivalence recommendations algorithm to configure a meal plan generation algorithm to create/update a meal, a 1-n meal plan for 1-n days, and shopping lists derived therefrom, based on a user's travel.

According to some embodiments, a user wanting to receive location based meal plan menus may operate a geofenced equivalence recommendations wizard with the meal plan generation application on the user's mobile device. Once the geofenced equivalence recommendations wizard is open on the user's mobile device, the wizard displays a few questions to the user to configure the geofenced equivalence recommendations algorithm. The wizard requests information regarding the type of food options source from which a user wishes to obtain food (e.g., prepared meals options (e.g., restaurants, delivery, etc.,) or food components (e.g., grocery stores)), the distance the user is willing to travel to obtain the foods whiles they are traveling, and the proximal food preferences and food brands a user may have available while they are traveling (e.g., traveling to Philadelphia, user may want to have a cheesesteak, or only want to have Dannon Yogurt purchased at a Safeway). The wizard takes the user responses and configures the meal plan generation application to display all the matching restaurants, grocery stores, and other food or meal access options in the geo tagged area to where the user will be traveling and/or the user's user device location. The user selects a day/meal range identifying the days/times that the user will be in the particular area, and then the meal plan generation algorithm generates the needed items and updates the menu. In some configurations, the system may allow food vendors to read information from the planned menu and allow the venders to set up delivery times or have meals ready for the user to pick up.

The wizard may also provide the user a number of food options to select from for that travel location allowing the user to select the best meal based on the preferences of the user and the available menu options.

The meal plan generation application may allow the user to select whatever meal they want from a restaurant's menu. If the user is selecting either a breakfast, morning snack, lunch, or afternoon snack, then the meal plan generation algorithm will re-calculate the dinner/evening meal/snack selections to get closer to the nutrient and kcal targets for that day.

The meal plan generation application may also generate the best three options for the user based on the preferences the user has in their profile. All three choices may result in the previously generated meal plan menu being updated such as, having the appropriate meals swapped out, recalculating future meals, etc.

One of skill in the art will realize that the methods and apparatuses of this disclosure describe prescribed functionality associated with a specific, structured graphical interface. Specifically, the methods and apparatuses, inter alia, are directed to a system and method for generating geofenced equivalence recommendations for a menu generating system. One of skill in the art will realize that these methods are significantly more than abstract data collection and manipulation.

Further, the methods provide a technological solution to a technological problem, and do not merely state the outcome or results of the solution. As an example, the system and method for generating geofenced equivalence recommendations for a menu generating system displays an updated food menu that accounts for a user's caloric and nutritional goals, and other relevant preferences, to a user through a user interface, based in part on the user's travel plans or their device location. Furthermore, the system and method for generating geofenced equivalence recommendations for a menu generating system reduces the load on the system by requiring fewer inputs from the user in creating the updated food menu, freeing system resources and thus improving the efficiency of the user interfacing device. This is a particular technological solution producing a technological and tangible result. The methods are directed to a specific technique that improves the relevant technology and are not merely a result or effect.

Additionally, the methods produce the useful, concrete, and tangible result of the system and method for generating geofenced equivalence recommendations for a menu generating system, thereby identifying each change as associated with its antecedent rule set.

Further, the methods are directed to a specifically-structured graphical user interface, where the structure is coupled to specific functionality. More specifically, the methods disclose a specific set of information to the user, rather than using conventional user interface methods to display a generic index on a computer.

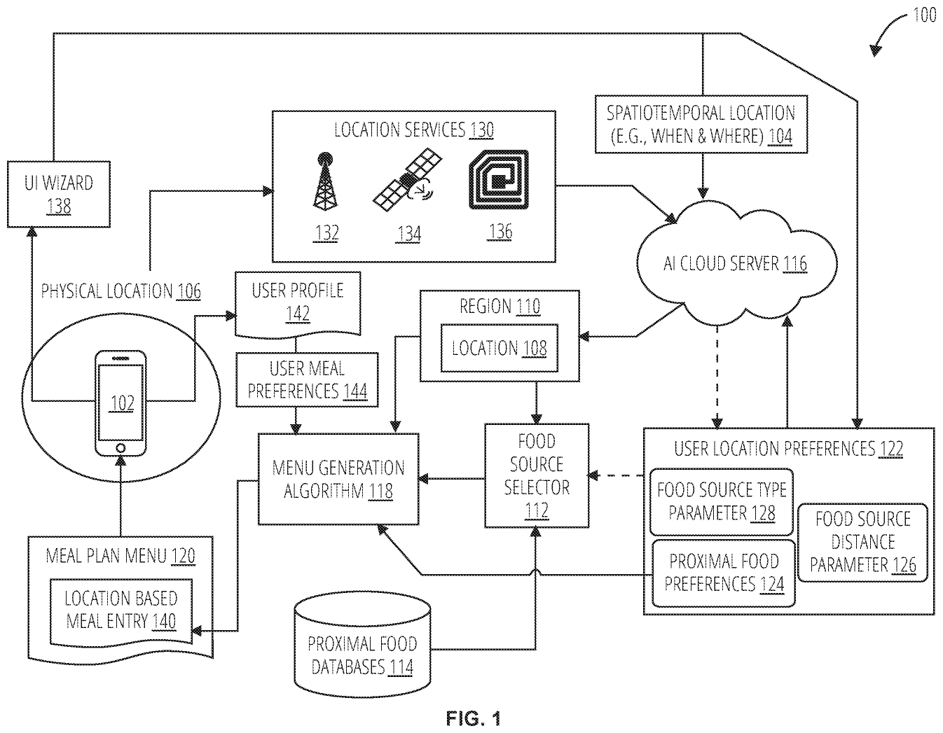

Referencing FIG. 1, a system 100 comprises a UI wizard 138 operated on a user device 102, a geofenced equivalence recommendations algorithm (AI cloud server) 116, a food source selector 112, a menu generation algorithm 118, and proximal food databases 114. The user device 102 shares a spatiotemporal location 104 and user location preferences 122 to a geofenced equivalence recommendations algorithm (AI cloud server) 116 operating on an AI cloud server through a UI wizard 138 operating with the menu generation system.

The geofenced equivalence recommendations algorithm (AI cloud server) 116 utilizes the spatiotemporal location 104 and the user location preferences 122 comprising a food source type parameter 128, a proximal food preferences 124, and a food source distance parameter 126, to identify a geographic region 110 and a geolocation 108 where the user device 102 will be. The food source selector 112 utilizes the geographic region 110, the geolocation 108, the proximal food preferences 124, the food source type parameter 128, and the food source distance parameter 126, to identify relevant local food sources from proximal food databases 114. The menu generation algorithm 118 is configured by a user meal preferences 144 from a user profile 142 associated with the user device 102, the geographic region 110, and the proximal food preferences 124 to select meals or food components from the relevant proximal food databases identified by the food source selector 112 in order to generate an at least one spatiotemporal location based meal entry 140 as part of a meal plan menu 120. The meal plan menu 120 is display to the user device 102. In some configurations, the user device 102 may provide it's physical location 106 to the geofenced equivalence recommendations algorithm (AI cloud server) 116 through location services 130 running on the user device 102. The location services 130 may include, but not limited to, location data provided by a wireless network 132 (e.g., cell towers, Wi-Fi, global positioning system (gps 134) data, and location information data from near field location beacons 136 (e.g., Bluetooth beacons, near field communication (NFC) beacons, etc., and location information captured by means of scanning or other visual data capture (e.g., scan of a QR code posted at a particular location). In some configurations the user meal preferences 144 may comprise food dislikes, food likes, food allergies or restrictions, meal and snack preferences (including preferred recipes), nutrient targets, weight or other personal health objectives, preferred food brands, and grocer or food distributor preferences, kcal target and meal presets. The linked user services may comprise a food tracking service and a grocery list service.

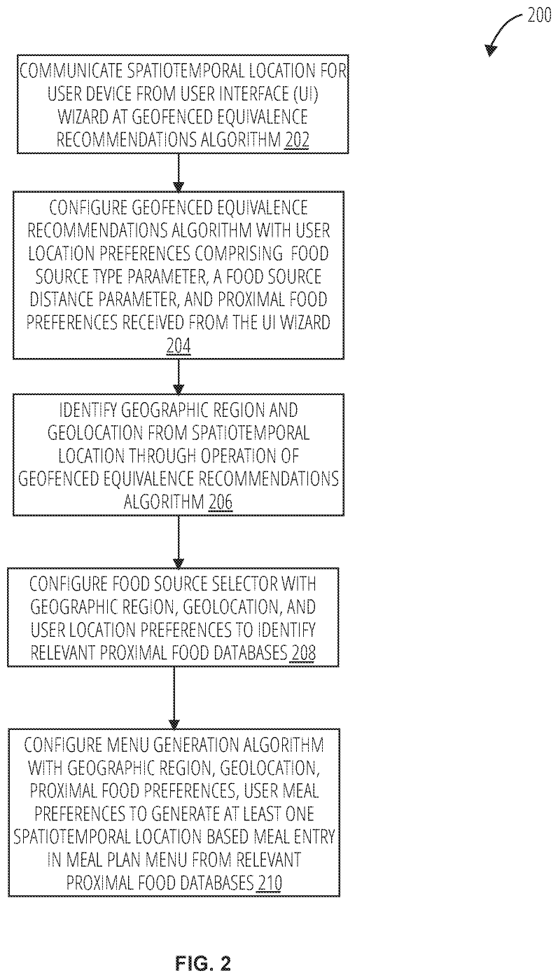

The system 100 may be operated in accordance with the process described in FIG. 2.

Referencing FIG. 2, a method 200 for generating geofenced equivalence recommendations for a menu generating system involves communicating spatiotemporal location from a user device accessing a user interface (UI) wizard at a geofenced equivalence recommendations algorithm (block 202). In block 204, method 200 configures the geofenced equivalence recommendations algorithm with user location preferences comprising a food source type parameter, a food source distance parameter, and proximal food preferences received from the UI wizard. In block 206, method 200 identifies a geographic region and a geolocation from the spatiotemporal location through operation of the geofenced equivalence recommendations algorithm. In block 208, method 200 configures a food source selector with the geographic region, the geolocation, and the user location preferences to identify relevant proximal food databases. In block 210, method 200 configures a menu generation algorithm with the geographic region, the geolocation, the proximal food preferences, and user meal preferences to generate at least one spatiotemporal location based meal entry in a meal plan menu from the relevant proximal food databases.

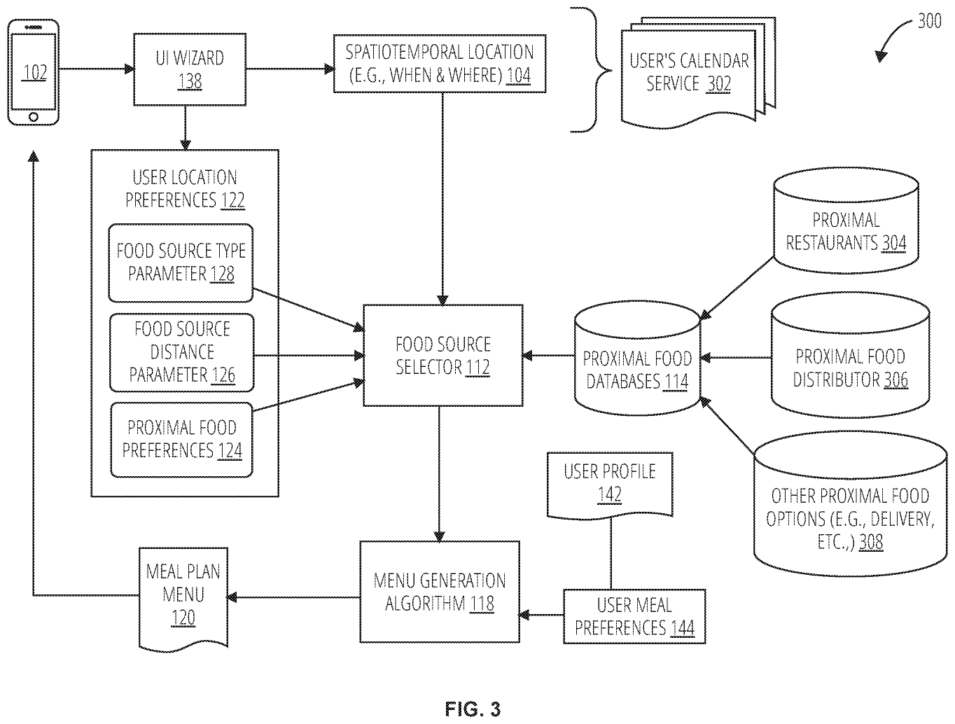

Referencing FIG. 3, a system 300 illustrates a geofenced equivalence recommendations algorithm receiving a spatiotemporal location 104 from a user device 102 by way of the UI wizard 138. The UI wizard 138 also provides the user location preferences 122 to configure the food source selector 112 to select relevant proximal food databases from a proximal food databases 114 comprising a proximal restaurant database 304, a proximal food distributor database 306, and an other local food options (e.g., brands) database 308. The system 300 may also receive the spatiotemporal location 104 from a user's calendar service 302. The food source selector 112 utilizes the food source type parameter 128, the food source distance parameter 126, and the proximal food preferences 124 to provide a menu generation algorithm 118 with the relevant proximal food databases and the user meal preferences 144 from the user profile 142 to generate a meal plan menu 120.

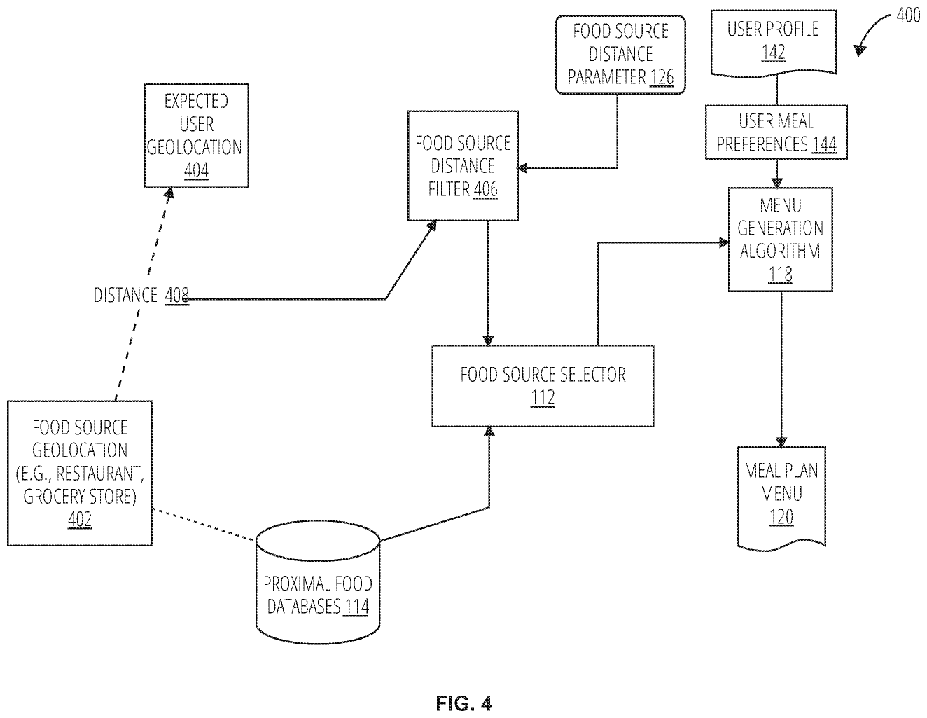

Referencing FIG. 4, a system 400 illustrates the selection of a local food source database from the proximal food databases 114 based on the distance 408 between the food source geolocation 402 and the expected user geolocation 404 through the use of a food source distance filter 406 configured by the food source distance parameter 126. The food source distance filter 406 configures the food source selector 112 to select local food source databases where the distance 408 is within the food source distance parameter 126 in order to provide the those databases to the menu generation algorithm 118 to generate a meal plan menu 120.

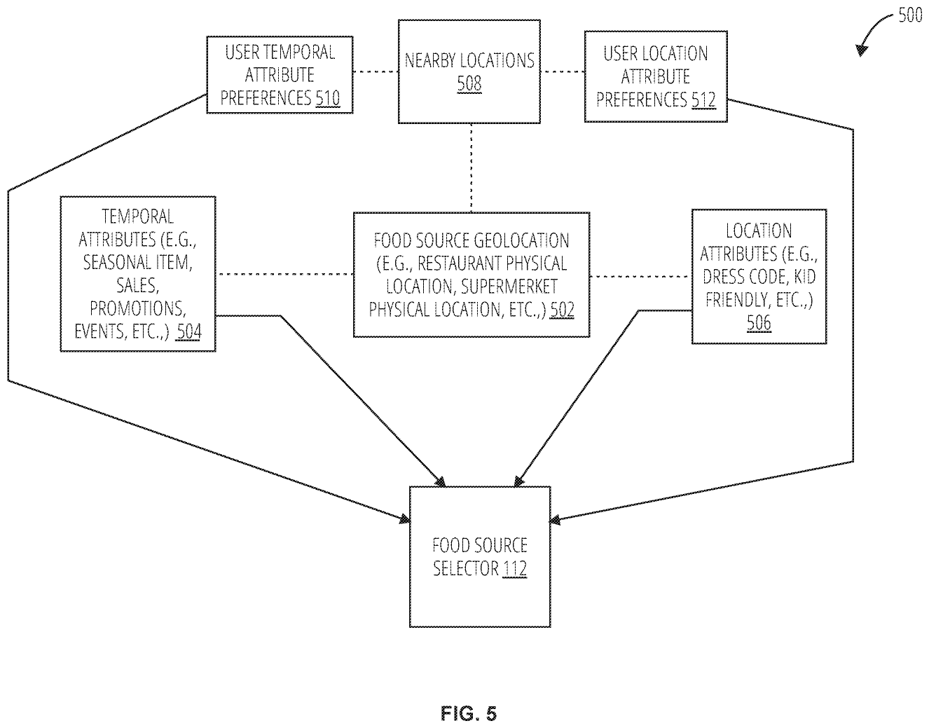

Referencing FIG. 5, a system 500 illustrates a configuration where in addition to filtering a food source geolocation 502 based on distance, (e.g., if it is a nearby locations 508) the food source geolocation 502 may be filtered based on user temporal attribute preferences 510 and user location attribute preferences 512 associated with it. For example, the food source geolocation 502 comprises temporal attributes 504 (e.g., seasonal item, sales, promotions, events, etc.,) and location attributes 506 (e.g., dress code, kid friendly, etc.,), a user may select to avoid locations that fall outside of their preferences. The user temporal attribute preferences 510 and the user location attribute preferences 512 may be utilized by the food source selector 112 do select from among relevant proximal food databases.

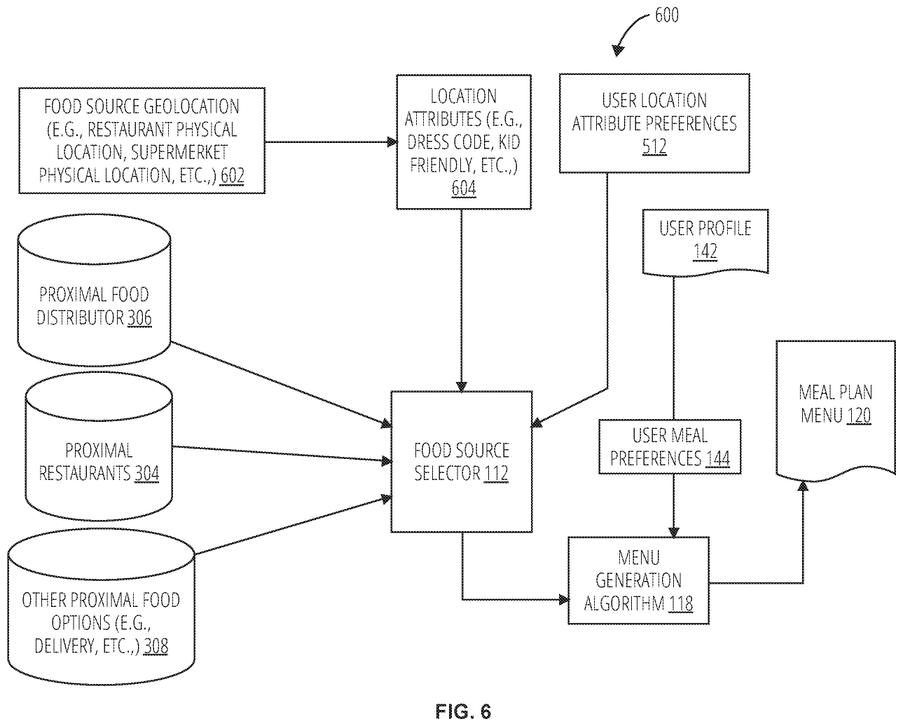

Referencing FIG. 6, a system 600 illustrates how the geofenced equivalence recommendations algorithm communicates the food source location attributes 604 of a food source geolocation 602 to help the food source selector 112 to select the relevant proximal food databases based on the user location attribute preferences 512.

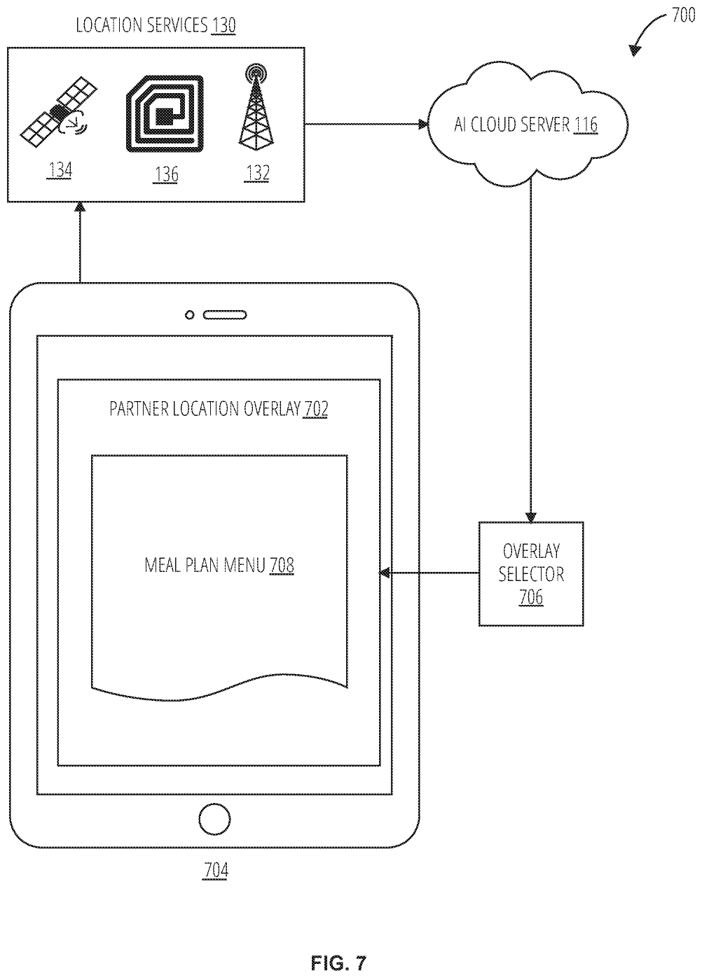

Referencing FIG. 7, a system 700 illustrates a user device 704 displaying a partner location overlay 702 with the meal plan menu 708 within a user interface. The partner location overlay 702 may be selected using an overlay selector 706 configured by the geofenced equivalence recommendations algorithm (AI cloud server) 116 based on the geolocation of the user device 704 provided by the location services 130.

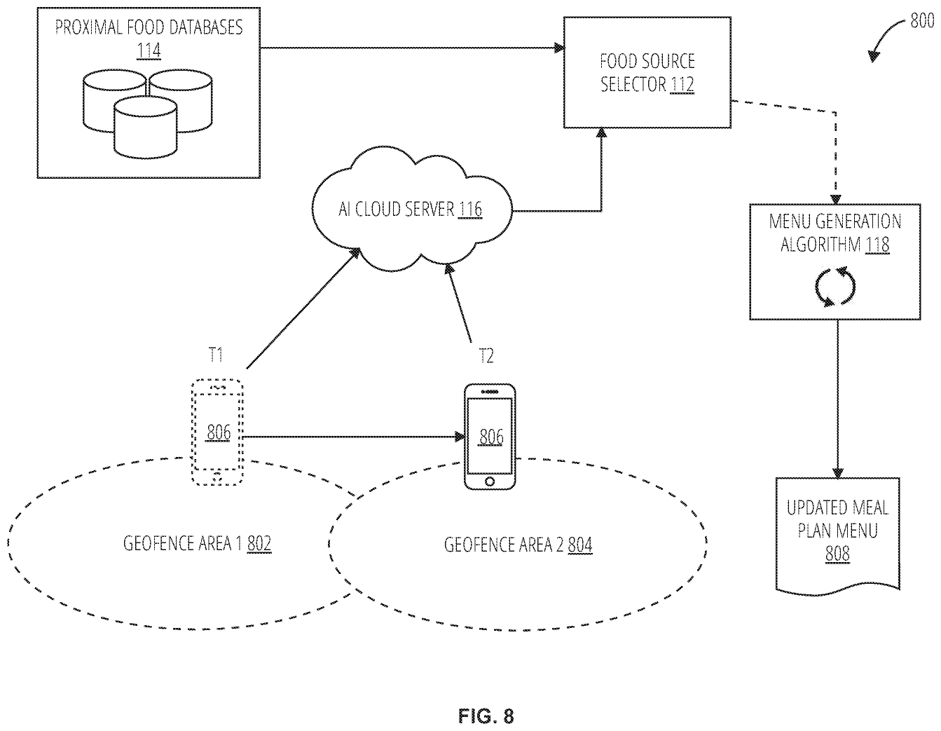

Referencing FIG. 8, a system 800 illustrates the process of generating an updated meal plan menu 808 based on the movement of the user's user device. At T1 the user device 806 is in geofence area 1 802 and communicates this information to the geofenced equivalence recommendations algorithm (AI cloud server) 116. When the user device 806 moves into geofence area 2 804 at T2, the geofenced equivalence recommendations algorithm (AI cloud server) 116 detects the change in the geofenced area and communicates the information to the food source selector 112 to select relevant proximal food databases for the menu generation algorithm 118. The menu generation algorithm 118 then generates an updated meal plan menu 808 with updated location information.

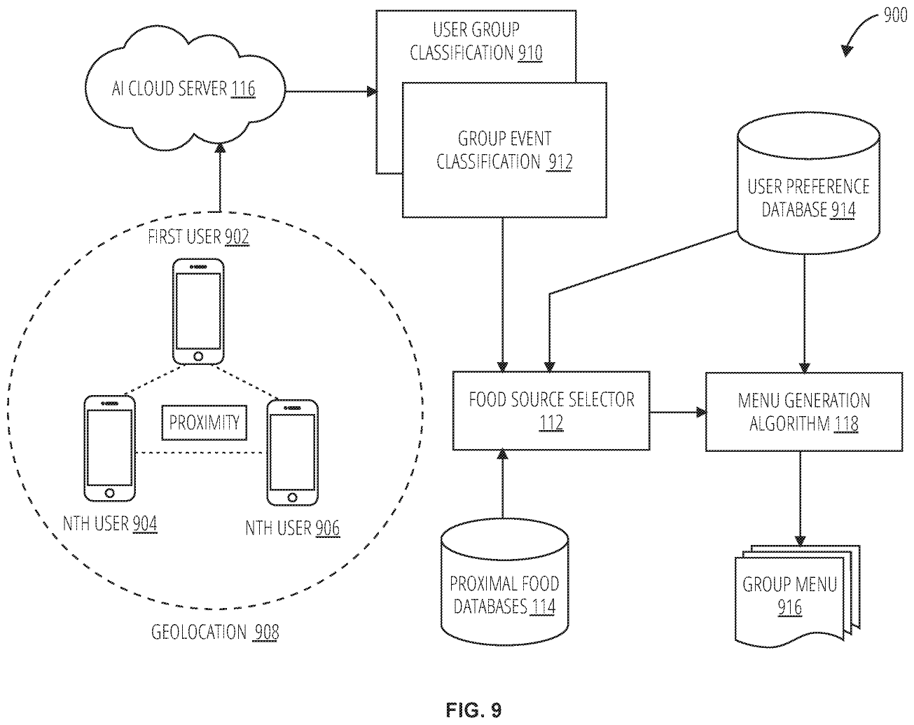

Referencing FIG. 9, a system 900 illustrates a configuration where a group menu 916 may be generated for a set of users based on their proximity to each other within a geolocation 908. For example, based on proximity, a geofenced equivalence recommendations algorithm (AI cloud server) 116 may determine that a first user 902, an nth user 904, and nth user 906, are in the same geolocation 908. The geofenced equivalence recommendations algorithm (AI cloud server) 116 may then identify a user group classification 910 to determine if an existing relationship exists between the users and if so determine if there is a group event classification 912. Based on the user group classification 910, and/or the group event classification 912, the user preferences from a user preference database 914, the food source selector 112 may select relevant proximal food databases to all the users to generate a group menu 916.

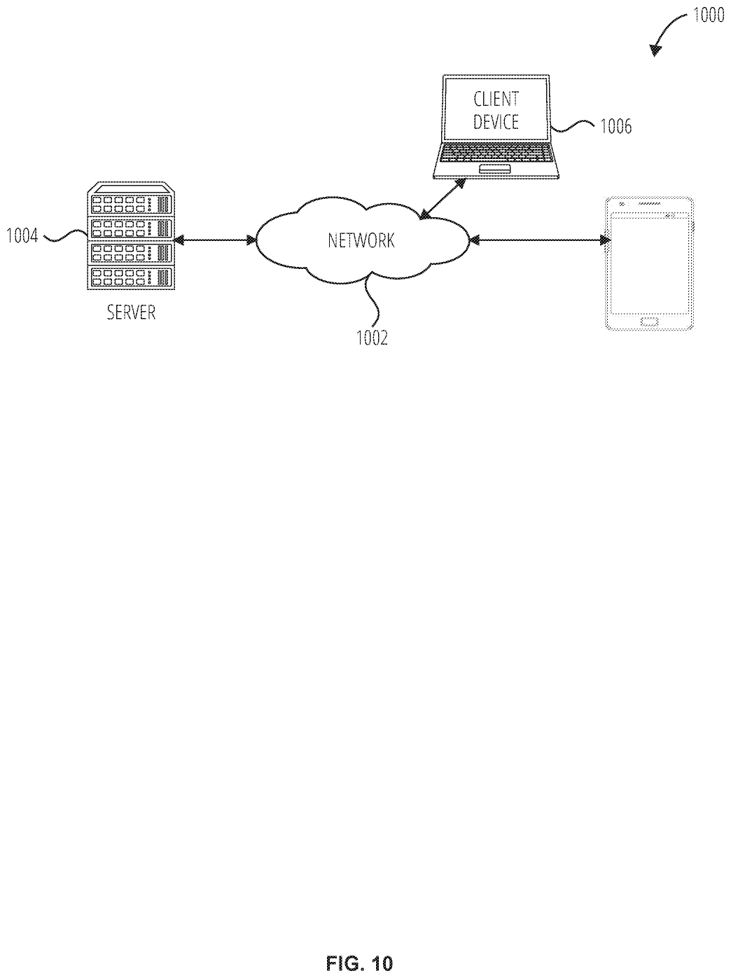

FIG. 10 illustrates a system 1000 in which a server 1004 and a client device 1006 are connected to a network 1002.

In various embodiments, the network 1002 may include the Internet, a local area network (“LAN”), a wide area network (“WAN”), and/or other data network. In addition to traditional data-networking protocols, in some embodiments, data may be communicated according to protocols and/or standards including near field communication (“NFC”), Bluetooth, power-line communication (“PLC”), and the like. In some embodiments, the network 1002 may also include a voice network that conveys not only voice communications, but also non-voice data such as Short Message Service (“SMS”) messages, as well as data communicated via various cellular data communication protocols, and the like.

In various embodiments, the client device 1006 may include desktop PCs, mobile phones, laptops, tablets, wearable computers, or other computing devices that are capable of connecting to the network 1002 and communicating with the server 1004, such as described herein.

In various embodiments, additional infrastructure (e.g., short message service centers, cell sites, routers, gateways, firewalls, and the like), as well as additional devices may be present. Further, in some embodiments, the functions described as being provided by some or all of the server 1004 and the client device 1006 may be implemented via various combinations of physical and/or logical devices. However, it is not necessary to show such infrastructure and implementation details in FIG. 10 to describe an illustrative embodiment.

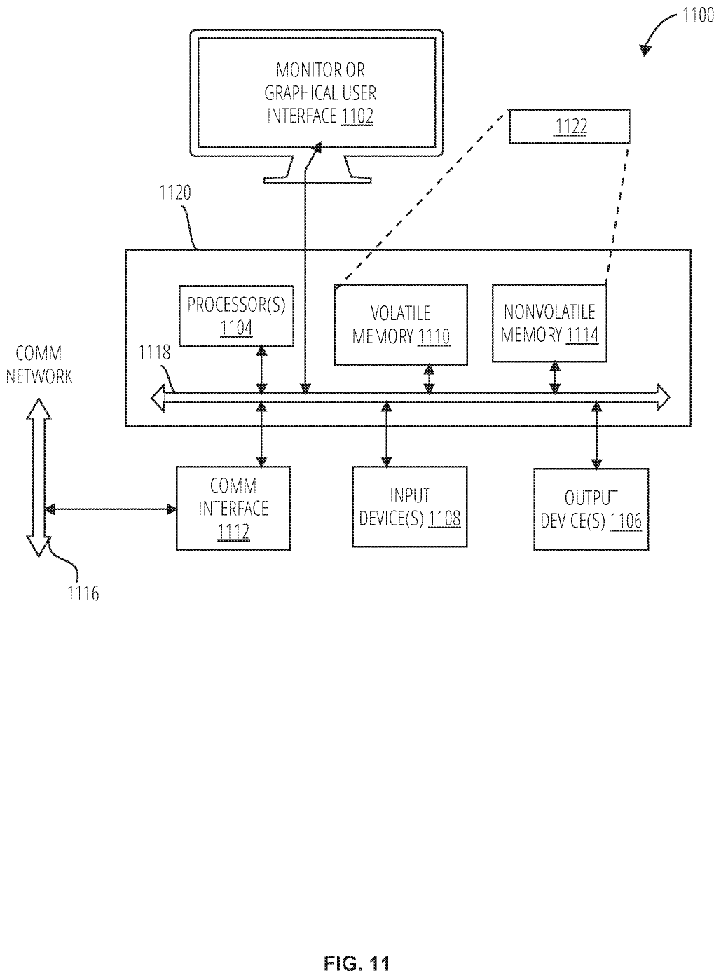

FIG. 11 is an example block diagram of a computing device 1100 that may incorporate embodiments of the present invention. FIG. 11 is merely illustrative of a machine system to carry out aspects of the technical processes described herein, and does not limit the scope of the claims. One of ordinary skill in the art would recognize other variations, modifications, and alternatives. In one embodiment, the computing device 1100 typically includes a monitor or graphical user interface 1102, a data processing system 1120, a communication network interface 1112, input device(s) 1108, output device(s) 1106, and the like.

As depicted in FIG. 11, the data processing system 1120 may include one or more processor(s) 1104 that communicate with a number of peripheral devices via a bus subsystem 1118. These peripheral devices may include input device(s) 1108, output device(s) 1106, communication network interface 1112, and a storage subsystem, such as a volatile memory 1110 and a nonvolatile memory 1114.

The volatile memory 1110 and/or the nonvolatile memory 1114 may store computer-executable instructions and thus forming logic 1122 that when applied to and executed by the processor(s) 1104 implement embodiments of the processes disclosed herein.

The input device(s) 1108 include devices and mechanisms for inputting information to the data processing system 1120. These may include a keyboard, a keypad, a touch screen incorporated into the monitor or graphical user interface 1102, audio input devices such as voice recognition systems, microphones, and other types of input devices. In various embodiments, the input device(s) 1108 may be embodied as a computer mouse, a trackball, a track pad, a joystick, wireless remote, drawing tablet, voice command system, eye tracking system, and the like. The input device(s) 1108 typically allow a user to select objects, icons, control areas, text and the like that appear on the monitor or graphical user interface 1102 via a command such as a click of a button or the like.

The output device(s) 1106 include devices and mechanisms for outputting information from the data processing system 1120. These may include the monitor or graphical user interface 1102, speakers, printers, infrared LEDs, and so on as well understood in the art.

The communication network interface 1112 provides an interface to communication networks (e.g., communication network 1116) and devices external to the data processing system 1120. The communication network interface 1112 may serve as an interface for receiving data from and transmitting data to other systems. Embodiments of the communication network interface 1112 may include an Ethernet interface, a modem (telephone, satellite, cable, ISDN), (asynchronous) digital subscriber line (DSL), FireWire, USB, a wireless communication interface such as Bluetooth or WiFi, a near field communication wireless interface, a cellular interface, and the like.

The communication network interface 1112 may be coupled to the communication network 1116 via an antenna, a cable, or the like. In some embodiments, the communication network interface 1112 may be physically integrated on a circuit board of the data processing system 1120, or in some cases may be implemented in software or firmware, such as “soft modems”, or the like.

The computing device 1100 may include logic that enables communications over a network using protocols such as HTTP, TCP/IP, RTP/RTSP, IPX, UDP and the like.

The volatile memory 1110 and the nonvolatile memory 1114 are examples of tangible media configured to store computer readable data and instructions to implement various embodiments of the processes described herein. Other types of tangible media include removable memory (e.g., pluggable USB memory devices, user device SIM cards), optical storage media such as CD-ROMS, DVDs, semiconductor memories such as flash memories, non-transitory read-only-memories (ROMS), battery-backed volatile memories, networked storage devices, and the like. The volatile memory 1110 and the nonvolatile memory 1114 may be configured to store the basic programming and data constructs that provide the functionality of the disclosed processes and other embodiments thereof that fall within the scope of the present invention.

Logic 1122 that implements embodiments of the present invention may be stored in the volatile memory 1110 and/or the nonvolatile memory 1114. Said logic 1122 may be read from the volatile memory 1110 and/or nonvolatile memory 1114 and executed by the processor(s) 1104. The volatile memory 1110 and the nonvolatile memory 1114 may also provide a repository for storing data used by the logic 1122.

The volatile memory 1110 and the nonvolatile memory 1114 may include a number of memories including a main random access memory (RAM) for storage of instructions and data during program execution and a read only memory (ROM) in which read-only non-transitory instructions are stored. The volatile memory 1110 and the nonvolatile memory 1114 may include a file storage subsystem providing persistent (non-volatile) storage for program and data files. The volatile memory 1110 and the nonvolatile memory 1114 may include removable storage systems, such as removable flash memory.

The bus subsystem 1118 provides a mechanism for enabling the various components and subsystems of data processing system 1120 communicate with each other as intended. Although the communication network interface 1112 is depicted schematically as a single bus, some embodiments of the bus subsystem 1118 may utilize multiple distinct busses.

It will be readily apparent to one of ordinary skill in the art that the computing device 1100 may be a device such as a smartphone, a desktop computer, a laptop computer, a rack-mounted computer system, a computer server, or a tablet computer device. As commonly known in the art, the computing device 1100 may be implemented as a collection of multiple networked computing devices. Further, the computing device 1100 will typically include operating system logic (not illustrated) the types and nature of which are well known in the art.

Terms used herein should be accorded their ordinary meaning in the relevant arts, or the meaning indicated by their use in context, but if an express definition is provided, that meaning controls.

“Circuitry” refers to electrical circuitry having at least one discrete electrical circuit, electrical circuitry having at least one integrated circuit, electrical circuitry having at least one application specific integrated circuit, circuitry forming a general purpose computing device configured by a computer program (e.g., a general purpose computer configured by a computer program which at least partially carries out processes or devices described herein, or a microprocessor configured by a computer program which at least partially carries out processes or devices described herein), circuitry forming a memory device (e.g., forms of random access memory), or circuitry forming a communications device (e.g., a modem, communications switch, or optical-electrical equipment).

“Firmware” refers to software logic embodied as processor-executable instructions stored in read-only memories or media.

“Hardware” refers to logic embodied as analog or digital circuitry.

“Logic” refers to machine memory circuits, non transitory machine readable media, and/or circuitry which by way of its material and/or material-energy configuration comprises control and/or procedural signals, and/or settings and values (such as resistance, impedance, capacitance, inductance, current/voltage ratings, etc.), that may be applied to influence the operation of a device. Magnetic media, electronic circuits, electrical and optical memory (both volatile and nonvolatile), and firmware are examples of logic. Logic specifically excludes pure signals or software per se (however does not exclude machine memories comprising software and thereby forming configurations of matter).

“Software” refers to logic implemented as processor-executable instructions in a machine memory (e.g. read/write volatile or nonvolatile memory or media).

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to a single one or multiple ones. Additionally, the words “herein,” “above,” “below” and words of similar import, when used in this application, refer to this application as a whole and not to any particular portions of this application. When the claims use the word “or” in reference to a list of two or more items, that word covers all of the following interpretations of the word: any of the items in the list, all of the items in the list and any combination of the items in the list, unless expressly limited to one or the other. Any terms not expressly defined herein have their conventional meaning as commonly understood by those having skill in the relevant art(s).

Various logic functional operations described herein may be implemented in logic that is referred to using a noun or noun phrase reflecting said operation or function. For example, an association operation may be carried out by an “associator” or “correlator”. Likewise, switching may be carried out by a “switch”, selection by a “selector”, and so on.

What is claimed is:

1. A method of generating geofenced equivalence recommendations for a menu generating system, the method comprising:
communicating spatiotemporal location for a user device from a user interface (UI) wizard at a geofenced equivalence recommendations algorithm;
configuring the geofenced equivalence recommendations algorithm with user location preferences comprising a food source type parameter, a food source distance parameter, and proximal food preferences received from the UI wizard;
identifying a geographic region and a geolocation from the spatiotemporal location through operation of the geofenced equivalence recommendations algorithm;
configuring a food source selector with the geographic region, the geolocation, and the user location preferences to identify relevant proximal food databases; and
configuring a menu generation algorithm with the geographic region, the geolocation, the proximal food preferences, and user meal preferences to generate at least one spatiotemporal location based meal entry in a meal plan menu from the relevant proximal food databases.

2. The method of claim 1 further comprising:
receiving the geolocation of the user device through the user device's location services at the geofenced equivalence recommendations algorithm; and
identifying the geographic region from the geolocation through operation of the geofenced equivalence recommendations algorithm.

3. The method of claim 1 further comprising receiving the spatiotemporal location from a user's calendar service linked with the menu generating system.

4. The method of claim 3, wherein receiving the spatiotemporal location from a user's calendar service comprises a future date.

5. The method of claim 1, wherein the user meal preferences comprise food dislikes, food likes, food allergies or restrictions, meal and snack preferences (including preferred recipes), nutrient targets, weight or other personal health objectives, preferred food brands, and grocer or food distributor preferences, kcal target and meal presets.

6. The method of claim 1, wherein the user meal preferences comprises one or more food acquisition options.

7. The method of claim 1, wherein the meal plan menu comprises multiple meals per day, and the menu generation algorithm is configured to generate two or more spatiotemporal location based meal entries into the meal plan.

8. The method of claim 7, wherein the spatiotemporal location for a user device is associated with a future location of the user device.

9. The method of claim 1, further comprising configuring the menu generation algorithm with caloric and nutritional goals for a user, and the spatiotemporal location based meal entry is generated, at least in part, on the caloric and nutritional goals.

10. The method of claim 1, further comprising generating at least three spatiotemporal location based meal entries and receiving a user selection of one or more of the spatiotemporal location based meal entries and including the selected one or more of the spatiotemporal location based meal entries into the meal plan menu.

11. The method of claim 1, further comprising communicating at least one of the spatiotemporal location based meal entries to a food vendor.

12. The method of claim 11, further comprising communication one or more of a delivery time, a delivery location, and a pick up time for the spatiotemporal location based meal entry.

13. The method of claim 1, further comprising receiving a nutritional goal for a user, and wherein the spatiotemporal location based meal entry is generated, at least in part, on the nutritional goal.

14. The method of claim 13, further comprising tracking nutritional information associated with user behavior, and wherein the spatiotemporal location based meal entry is modified, at least in part, on the user behavior and the nutritional goal.

15. A method of generating a meal plan, comprising:
determining a spatiotemporal location for a user device;
determining a geographic region and a geolocation from the spatiotemporal location;
determining a food source based, at least in part, on the geographic region;
determining caloric goals and nutritional goals for a user;
generating a meal entry based, at least in part, on the geographic region, the food source, the caloric goals and the nutritional goals.

16. The method of claim 15, wherein the spatiotemporal location for the user device is based upon a future location of the user device.

17. The method of claim 15, wherein the food source is one or more of a restaurant, a grocery store, or a food delivery service.

18. The method of claim 17, further comprising generating a shopping list for one or more food components in stock at the grocery store.

19. A method of generating a geofenced menu, comprising:
determining a location of a user;
determining nutritional goals of the user
generating, by a menu generation algorithm and based at least in part on the nutritional goals of the user, a meal plan menu comprising one or more meals.

20. The method of claim 19, further comprising:
determining that the location of the user has changed to a second location; and
modifying, by the menu generation algorithm and based at least in part on the nutritional goals and the second location of the user, the meal plan menu.
